(19) United States
(12) Patent Application Publication
(10) Pub. No.: US 2003/0108499 A1
(43) Pub. Date: Jun. 12, 2003

(54) Deodorant compositions

(75) Inventors: Elizabeth Jane Smith, Merseyside (GB); Andrew Sjaak Landa, Merseyside (GB); Stephen Anthony Makin, Lancashire (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/198,435

(22) Filed: Jul. 17, 2002

(30) Foreign Application Priority Data
Jul. 18, 2001 (GB) 0117535.5

(51) Int. Cl. A61K 7/32

(52) U.S. Cl. 424/65

(57) ABSTRACT

The present invention describes a deodorant composition comprising a suspension of a finely-divided particulate transition metal chelator in a silicone fluid carrier material that lacks a significant amount of astringent antiperspirant salts.

### FIELD OF INVENTION

This invention relates to the field of anti-microbial and deodorant compositions. In addition, this invention is concerned with achieving a deodorancy benefit upon the surface of the human body and in close proximity thereto. The compositions and methods involve finely-divided particulate transition metal chelator suspended in a silicone fluid carrier material. The compositions and methods of the invention are generally of greatest benefit when used on the most malodorous areas of the body, for example the underarm areas or feet.

### BACKGROUND

Anti-microbial and deodorant compositions may function by a variety of means. Such compositions may function by significantly reducing microbial numbers either by reducing perspiration or by directly affecting the micro-organisms on the body surface as represented by skin. It is with this latter mechanism of action that this invention is largely concerned.

Most anti-microbial and deodorant compositions reduce the number of viable micro-organisms on the surface of the skin. It is well known that sweat is usually odourless until it has been degraded by the skin microflora. Typical deodorants include ethanol and triclosan (2′,4,4′-trichloro, 2-hydroxy-diphenyl ether) which is a well known anti-microbial agent. However, the deodorising effect obtained with such deodorants wears off with the passage of time and the microflora progressively recover their numbers. In addition, compositions that function in the above way are not generally perceived as giving a satisfactory antiperspirancy benefit.

There is, therefore, a need for effective and long lasting deodorant compositions on the market. The problem to be solved is not simply reducing microbial numbers on the body surface; equally important is maintaining low microbial numbers (particularly low bacterial numbers) on the body surface (particularly in the most malodorous areas, e.g. the axillae and feet).

In addition, the deodorant compositions that meet the above need must have good physical/chemical stability and preferably be easy to produce and to use. A further requirement of a topically applied composition is comfort in use. This is particularly true when the product is to be left on the surface of the body for an extended period, as with topically applied long-lasting anti-microbial and deodorant compositions. Comfort in use may include both minimisation of any discomfort on application and wear, and positive sensory benefits.

Deodorant compositions comprising chelators are described in our recent PCT patent applications PCT/EP01/00111, PCT/EP01/00112 and PCT/EP01/00118, and GB patent application 0024689.2. These patent applications disclose the hypothesis that certain chelators can effectively inhibit the up-take of essential transition metal ion nutrients by microbes on the skin surface, thereby minimising their growth.

U.S. Pat. No. 4,356,190 (Personal Products Co.) discloses the use of selected aminopolycarboxylic acid compounds for inhibiting the formation of short chain fatty acids by Corynebacterium on the skin surface.

WO 97/44006 (Ciba Speciality Chemicals Holding, Inc.) claims the use of nitrogen-containing complexing agents for the anti-microbial treatment of the skin and of textile fibre materials.

WO 99/53892 (Miller) discloses depilatory compositions comprising chelating agent and a topical carrier that may include silicone oil.

U.S. Pat. No. 5,433,943 (Osipow et al) discloses antiperspirant and deodorant compositions comprising silicone fluid, chelator, and gelatin (as a water absorber).

### SUMMARY OF THE INVENTION

According to a first aspect of the present invention, there is provided a deodorant composition comprising a suspension of a particulate transition metal chelator in a silicone fluid carrier material, characterised in that the transition metal chelator has a weight average particle size of between 10 μm and 100 μm and in that the deodorant composition excludes a significant amount of astringent antiperspirant salts.

According to a second aspect of the present invention, there is provided a method of achieving a deodorancy benefit comprising the application to the human body or to an article wearable in close proximity thereto, of a composition according to the aforementioned first aspect of the invention.

According to a third aspect of the present invention, there is provided a method for the manufacture of a deodorant composition, said method comprising the suspension of a particulate transition metal chelator of weight average particle size between 10 μm and 100 μm in a silicone fluid carrier material, without addition of a significant amount of astringent antiperspirant salts to said deodorant composition.

### DETAILED DESCRIPTION

The method of controlling malodour offered by the present invention is particularly useful because the benefit can extend for many hours, for example 10 hours, 24 hours, or even longer, after application of the product. This can represent an extended deodorancy benefit; that is to say, extended inhibition of the generation of odour on the human body or on closely associated articles.

Articles wearable in close proximity to the human body may be protected from malodour generation by the present invention. Such articles include any garments worn next to the skin, for example stockings and socks, and also shoes and other items of footwear. The deodorant composition may be applied directly to the aforementioned articles, but is more commonly applied to the human body surface, particularly the more odiferous regions of the human body such as the axillae and feet.

The deodorant compositions of the invention may take any form and may be applied by any means. Application of liquid compositions may be by absorption onto a carrier matrix like paper, fabric, or sponge and application by contacting said carrier matrix with the surface to be treated.

Alternatively, liquid compositions may be applied directly by using conventional roll-on or spray applicators. Solid or semi-solid compositions may be applied by direct contact or may be dissolved or dispersed in a liquid medium prior to application. Application may also comprise a combination of any two or more of the above techniques.

The benefits obtained with the compositions of the invention may include any of the following: high deodorant efficacy, long lasting deodorancy, good perceived antiperspirancy, good physical/chemical stability, ease of manufacture, ease of use by consumers, and, on topical application to the human body, good sensory properties, that is to say, high comfort in use.

Particularly important for the consumer are the benefits of high deodorant efficacy and good sensory properties. Without wishing to be bound by theory, it is hypothesised that the small particle size of the chelator and the lack of a significant amount of astringent antiperspirant salt enhances the speed and/or extent of dissolution of the particulate transition metal chelator on contact with a moist surface, such as the surface of the human body. The enhanced dissolution enables the chelator to rapidly and/or extensively come into intimate contact with the microbes upon the surface being treated. The specified particle size of the chelator suspension, together with the presence of the silicone fluid, may also aid the good sensory properties of the compositions of the invention, for example the benefit of low grittiness, which is of particular use when the composition is applied directly to the surface of the human body.

The absence of a significant amount of astringent antiperspirant salt may also aid the good sensory properties (some people being sensitive to such salts); surprisingly, this benefit may be achieved without loss of the perceived antiperspirancy benefit.

It is preferred that the compositions of the invention are essentially anhydrous; that is to say, it is preferred that they comprises less than 10%, in particular less than 5%, and especially less than 1% by weight of water, excluding any volatile propellant that may be present.

The particulate transition metal in the compositions of the invention is in a solid form. Preferred transition metal chelators have affinity for iron (III), preferably high affinity for iron (III); that is to say, a binding constant for iron (III) of greater than 10¹⁰, or, for optimum performance, greater than 10²⁶. The ‘iron (III) binding constant’ referred to above is the absolute stability constant for the chelator-iron (III) complex. Such values are independent of pH and are measured on the most anionic, fully deprotonated form of the chelator. Measurements can be made potentiometrically, and in a number of other ways. Full details of suitable methods can be found in “Determination and Use of Stability Constants”, A. E. Martell and R. J. Motekaitis (VCH, New York, 1989). Tables of applicable values may be found in numerous sources, for example “Critical Stability Constants”, R. M. Smith and A. E. Martell (Plenum Pub. Corp., 1977).

The chelators used in the present invention preferably have acid forms with at least two ionisable acid groups. The acid groups are preferably carboxylic, although phosphonic, sulphonic, phosphinic, or any mixture of these groups may be present.

It is preferred that chelators having acid groups are used in their acid form or as acid salts (i.e. partially neutralised); it is more preferred that such chelators be used in their acid form. When chelator salts or acid salts are employed, suitable counter-ions are inorganic cations such as alkali metals, including sodium and potassium, or alkaline earth metals, including magnesium, or organic cations such as protonated or quaternised amines. A particularly preferred counter-ion is sodium.

Preferred chelators with carboxylic acid groups are aminopolycarboxylate compounds. The acid forms of preferred aminopolycarboxylate compounds include ethylenediaminetetraacetic acid (EDTA), trans-1,2-diaminocyclohexane-N,N,N′,N′-tetraacetic acid (CDTA), and ethylenediaminedisuccinic acid (EDDS). More preferred aminopolycarboxylate chelators have the acid forms diethylenetriaminepentaacetic acid (DTPA), triethylenetetraaminehexaacetic acid (TTHA), and ethylenebis[2-(2-hydroxyphenyl)glycine] (EDDHA). Chelators having the acid form DTPA are especially preferred.

The transition metal chelator is preferably used at a level of at least 0.2% by weight of the composition, excluding any volatile propellant also present. In particular, the chelator is used at a level of from 0.5% to 5% and especially at a level of from 0.65% to 3% by weight of the composition. When mixtures of chelators are employed, the aforementioned preferred levels refer to the total amount of chelator present.

The silicone fluid carrier material employed may be selected from any of those known in the art. Volatile liquid silicones, that is to say, liquid polyorganosiloxanes are preferred. To class as “volatile”, such material should have a measurable vapour pressure at 20□C or 25□C. Typically, the vapour pressure of a volatile silicone lies in a range from 1 or 10 Pa to 2 kPa at 25° C.

It is desirable to include a volatile silicone because it gives a “drier” feel after the composition is applied to skin.

Volatile polyorganosiloxanes can be linear or cyclic or mixtures thereof. Preferred cyclic siloxanes include polydimethylsiloxanes, particularly those containing from 3 to 9 silicon atoms, preferably not more than 7 silicon atoms, and most preferably from 4 to 6 silicon atoms, otherwise often referred to as cyclomethicones. Preferred linear siloxanes include polydimethylsiloxanes containing from 3 to 9 silicon atoms. The volatile siloxanes normally by themselves exhibit viscosities of below 10⁻⁵ m²/sec (10 centistokes), and particularly above 10⁻⁷ m²/sec (0.1 centistokes); the linear siloxanes normally exhibiting a viscosity of below 5×10⁻⁶ m²/sec (5 centistokes). The volatile silicones can also comprise branched linear or cyclic siloxanes such as the aforementioned linear or cyclic siloxanes substituted by one or more pendant —O—Si(CH₃)₃ groups. Examples of commercially available silicone fluids include fluids having grade designations 344, 345, 244, 245, and 246, from Dow Corning Corporation; Silicone 7207 and Silicone 7158 from Union Carbide Corporation; and SF1202 from General Electric.

Non-volatile silicone fluids include polyalkyl siloxanes, polyalkylaryl siloxanes, and polyethersiloxanes copolymers. Examples include dimethicones and dimethicone copolyols. Commercial examples include Dow Corning silicone fluids 556, 704, and the 200 series.

Mixtures of silicone fluids may also be used. The total amount of silicone fluid present in compositions of the invention is preferably from 3% to 95% and especially from 6% to 70% by weight of the composition. In aerosol compositions also comprising a volatile propellant, the preferred level is from 3% to 50% and especially from 6% to 25% by weight of the composition.

The compositions of the invention exclude a significant amount astringent antiperspirant salts. In this context, an astringent antiperspirant salt is any of those astringent salts that have been commonly used in the art for gaining an antiperspirancy benefit. The designation “salts” includes the case when only one such salt is present. Astringent antiperspirant salts are disclosed in “Antiperspirants and Deodorants”, Ed. K. Laden, 1999, Marcel Dekker, New York, and include include aluminium, zirconium and aluminium/zirconium halides and halohydrate salts, such as chlorohydrates, and their complexes with amino acids such as glycine. The astringent salts may be considered to be present in a significant amount when they deliver an antiperspirancy benefit on topical application of the composition to the human body. It is desirable that astringent antiperspirant salts comprise less than 5% by weight, preferably less than 1% by weight of the composition.

In a preferred aspect of the invention, the deodorant compositions comprise little, if any, additional components (ie. components other than the chelator or the silicone fluid) having a water absorbency greater than 2.5 g/g.

Water absorbency may be determined by the method detailed in Example 3. Thus, preferred compositions of the invention comprise an amount by weight of additional components having a water absorbency of greater than 2.5 g/g that is less than the amount by weight of transition metal chelator; particularly preferred are compositions wherein the ratio by weight of transition metal chelator to additional components having a water absorbency of greater than 2.5 g/g is greater than 3:2, especially when this ratio by weight is greater than 2:1. The above preferences also apply to the total additional components having a slightly lower water absorbency, for example components having a water absorbency of greater than 2.0 g/g or even greater than 1.5 g/g. Having relatively low amounts of materials of this kind aids fast and effective dissolution of the chelator when the composition comes into contact with a moist surface (vide supra)

In addition to the silicone fluid carrier material, non-silicone hydrophobic liquids may be used. Such materials include mineral oils, hydrogenated polyisobutene, polydecene, paraffins, isoparaffins of at least 10 carbon atoms, aliphatic or aromatic ester oils (eg. isopropyl myristate, lauryl myristate, isopropyl palmitate, diisopropyl sebecate, diisopropyl adipate, or C₈ to C₁₈ alkyl benzoates), and polyglycol ethers, for example polyglycol butanol ethers.

Relatively hydrophilic liquids may also be used in the compositions of the invention, for example short chain (C2-C4) alcohols, like ethanol. Particularly preferred materials are those that can act as humectants or emollients, for example glycerol and propylene glycol.

Conventional organic anti-microbial agents may also be advantageously employed in the methods and compositions of the present invention. Levels of incorporation are preferably from 0.01% to 3% and especially from 0.03% to 0.5% by weight of the of the composition. Most of the classes of agents commonly used in the art can be utilised. Preferred additional organic anti-microbials are bactericides, for example quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred additional anti-microbials for use in the compositions of the invention are polyhexamethylene biguanide salts; 2′,4,4′-trichloro,2-hydroxy-diphenyl ether (triclosan); and 3,7,11-trimethyldodeca-2,6,10-trienol (farnesol).

Anti-oxidants may also be advantageously employed in the compositions of the invention. Such materials may comprise a tert-butylphenol group, preferably a di-tert-butylphenol group. Examples include BHA (butylated hydroxyanisole), BHT (butylated hydroxytoluene), 2,2′-ethylidenebis(4,6-di- tert-butylphenol), and pentaerythritol tetrakis(3-(3,5-di-tert-butyl-4-hydroxyphenyl)propionate). BHT is especially preferred. Such materials are typically used at a level of at between 0.05% and 5% by weight, preferably at a level of 0.075% to 2.5%, and especially at a level of 0.1% to 1% by weight of the composition.

Structurants and emulsifiers are further additional components of the compositions of the invention that are highly desirable in certain product forms. Structurants, when employed, are preferably present at from 1% to 30% by weight of the composition, whilst emulsifiers are preferably present at from 0.1% to 10% by weight of the composition. Suitable structurants include cellulosic thickeners such as hydroxy propyl cellulose and hydroxy ethyl cellulose, cellobiose esters, and dibenzylidene sorbitol. Other suitable structurants include sodium stearate, stearyl alcohol, cetyl alcohol, cetearyl alcohol, 2-octyldodecanol, hydrogenated castor oil, synthetic waxes, paraffin waxes, hydroxystearic acid, dibutyl lauroyl glutamide, alkyl silicone waxes, β-sitosterol/γ-oryzanol, and silica. Suitable emulsifiers include steareth-2, steareth-20, steareth-21, ceteareth-20, glyceryl stearate, PEG-20 stearate, and dimethicone copolyol.

Further emulsifiers/surfactants desirable in certain compositions of the invention are perfume solubilisers and wash-off agents. Examples of the former include PEG-hydrogenated castor oil, available from BASF in the Cremaphor RH and CO ranges, preferably present at up to 1.5% by weight, more preferably 0.3 to 0.7% by weight. Examples of the latter include poly(oxyethylene) ethers.

A fragrance material is a further desirable component in the compositions of the invention. Suitable materials include conventional perfumes, such as perfume oils and also include so-called deo-perfumes, as described in EP 545,556 and other publications. These latter materials may also qualify as additional organic anti-microbial agents. Levels of incorporation are preferably up to 4% by weight, particularly from 0.1% to 2% by weight, and especially from 0.7% to 1.7% by weight. Synergies can exist between the essential components the invention and certain fragrance components—long-lasting odour control being the result.

It should be noted that certain components of compositions perform more than one function. Such components are particularly preferred additional ingredients, their use often saving both money and formulation space. Examples of such components include ethanol, isopropyl myristate, and the many components that can act as both structurants and sensory modifiers, for example silica.

Further additional components that may also be included are colourants and preservatives, for example C₁-C₃ alkyl parabens.

Aerosol compositions represent a preferred embodiment of the present invention. Such compositions generally comprise a volatile propellant. The level of incorporation of the volatile propellant is typically from 30 to 99 parts by weight, and particularly from 50 to 95 parts by weight. Non-chlorinated volatile propellant may be used, in particular liquefied hydrocarbons or halogenated hydrocarbon gases (particularly fluorinated hydrocarbons such as 1,1-difluoroethane and/or 1-trifluoro-2-fluoroethane) that have a boiling point of below 10° C. and especially those with a boiling point below 0° C. Liquefied hydrocarbon gases are preferred in certain embodiments of the invention, especially C₃ to C₆ hydrocarbons, including propane, isopropane, butane, isobutane, pentane and isopentane and mixtures of two or more thereof. The most preferred propellants are isobutane, isobutane/isopropane, isobutane/propane and mixtures of isopropane, isobutane and butane.

Other propellants that may be contemplated include alkyl ethers, such as dimethyl ether or compressed non-reactive gasses such air, nitrogen or carbon dioxide.

A further component desirably present in aerosol and roll-on compositions according to the invention is a suspending agent. Such agents are typically used at levels of from 0.1 to 10%, in particular 0.25 to 5%, and especially from 0.5 to 3% by weight of the composition. Examples of such materials include organo-modifed clays, in particular organo-modified bentonites and hectorites. Quaternium-18 bentonite and quaternium-18 hectorite are especially preferred for this function. Propylene carbonate is a preferred additional ingredient when a suspending agent is employed. Propylene carbonate may be used at a level of from 0.05% to 5% and particularly at a level of from 0.2 to 0.3% by weight of the composition.

Methods of Manufacture

The method a manufacture of compositions according to the invention involves the suspension of a particulate transition metal chelator of weight average particle size between 10 μm and 100 μm in a silicone fluid carrier material, without addition of a significant amount of astringent antiperspirant salts to said deodorant composition. It is preferred that the chelator is stirred into the carrier fluid. When a suspending agent is employed, it is preferred that it is added to the carrier fluid before the chelator, preferably whilst shearing.

### EXAMPLES

Throughout the examples, comparative examples are designated by letter codes and amounts refer to parts by weight, unless otherwise indicated.

### Example 1

The compositions detailed in Table 1 were prepared in the following manner. The Bentone 38V was gradually added to the DC 245 whilst shearing at a speed of ca. 8000 rpm. After approximately 10 minutes, the propylene carbonate was added dropwise. After a further 5 minutes, shearing was stopped and the DTPA was slowly stirred into Example 1. After 5 minutes further mixing, the resulting base compositions were transferred to tin-plate aerosol cans and the propellant was added. The cans were sealed and fitted with a suitable actuator to enable effective spraying of the product.

TABLE 1
Compositions and Deodorancy Performance

| | | Example A | Example 1 |
|---|---|---|---|
| Compositions | Component: | | |
| | DC 245¹ | 12.3 | 11.3 |
| | Bentone 38V² | 0.5 | 0.5 |
| | Propylene | 0.2 | 0.2 |
| | carbonate | | |
| | DTPA³ | | 1.0 |
| | CAP 40⁴ | 87 | 87 |
| Mean malodour | 5 hours⁵ | 1.73 | 1.45 |
| result after: | 24 hours⁶ | 2.13 | 1.64 |

¹Cyclomethicone silicone carrier fluid, ex Dow Corning.
²Quaternium-18 hectorite suspending agent, ex Rheox.
³Diethylenetriamine pentaacetic acid chelator, of weight average particle size of between 10 μm and 45 μm.
⁴Propellant; proprietary mixture of butane, isobutane, and propane, ex Calor.
⁵Difference required for significance at the 99% level: 0.12; actual difference observed: 0.28.
⁶Difference required for significance at the 99% level: 0.12; actual difference observed: 0.49.

These results clearly indicate the good deodorancy performance of compositions of the invention, particularly after many hours.

The malodour results shown in Table 1 were obtained using a panel of 50 individuals who had been instructed to use control ethanolic deodorant products during the week prior to the test. At the start of the test, panellists were washed with unfragranced soap and different products (1.20g dose) were applied to each axilla. (Product application was randomised to take into account any left/right bias). Panellists were instructed not to consume spicy food or alcohol, and not to wash under their own axillae, during the duration of the test. At least three expert assessors determined the intensity of axillary malodour after the times indicated, scoring the intensity on a scale of 1-5. After each 24 hour assessment, the panellists were re-washed, and products re-applied, as above. The procedure was repeated 4 times. At the end of the test the data were analysed using standard statistical techniques.

### Example 2

The compositions indicated in Table 2 were prepared in a similar manner to those of Table 1.

TABLE 2
Further Compositions

| Component: | Example B | Example 2 |
|---|---|---|
| DC 245 | 13.1 | 11.3 |
| Bentone 38V | 1.0 | 0.5 |
| DTPA | | 1.0 |
| AACH¹ | 10.0 | |
| Perfume | 1.0 | 1.0 |
| CAP 40 | 74.9 | 86.2 |

¹aluminium chlorohydrate, A296 ex Giulini.

The compositions indicated in Table 2 were evaluated in a large consumer test, each product being tested by 159 individuals. Example 2 was found to have parity to Example B (a standard antiperspirant composition) on the following attributes:

kept me dry all day;

effective against keeping me dry;

effective against body malodour.

These results indicate the good perceived antiperspirancy and deodorancy benefits of the compositions of the invention, despite their lack of astringent antiperspirant active.

### Examples 3 and 4

These examples contrast the dissolution characteristics of a chelator in various mixtures.

The mixtures indicated in Table 3 were placed on a filter paper and 100 μl quantities of water were added over 1 minute intervals. The results given indicate that dissolution of the chelator in Examples 3 and 4 occurred more quickly and effectively than the chelator in Example 3A, illustrating the benefit of having a relatively low amount of additional components having a water absorbency of greater than 2.5 g/g.

TABLE 3
Dissolution Characteristics
(amounts indicated are in mg)

| | Example 3 | Example 4 | Example 3A |
|---|---|---|---|
| Components: | | | |
| EDTA trisodium salt¹ | 670 | 810 | 446 |
| Bentone 38V² | 330 | — | — |
| IPM³ | — | 192 | 107 |
| Talc⁴ | — | — | 446 |
| Water permeation: | | | |
| after 1300 μl: | 360 | 345 | 0 |
| after 1500 μl: | 560 | 995 | 90 |

¹Weight average particle size between 10 μm and 63 μm.
²Water absorbency: 2.4 g/g.
³Isopropyl myristate.
⁴Suprafino talc, ex Cyprus Industrial Minerals, water absorbancy: 2.8 g/g.

Water absorbencies were determined by placing 1 g of material on a filter paper and adding 100 μl (0.1 g) quantities of water, over 1 minute intervals, until water permeated through; the water absorbency being taken as the maximum amount of water added prior to permeation.

### Examples 5 to 13

The Examples given in Table 4 represent aerosol compositions, and those given in Table 5 represent non-aerosol compositions, which may be made in accordance with the invention.

TABLE 4
Aerosol Compositional Examples

| | Example: | | | | |
|---|---|---|---|---|---|
| Component: | 5 | 6 | 7 | 8 | 9 |
| DTPA | 0.20 | 0.50 | 0.65 | 3.00 | 5.00 |
| Bentone 38V | 0.50 | 0.50 | 0.50 | 1.00 | 1.00 |
| Propylene | 0.20 | 0.20 | | 0.30 | 0.30 |
| carbonate | | | | | |
| Perfume | 1.00 | 1.0 | 0.90 | 1.00 | 0.70 |
| DC 200¹ | | 0.30 | | | |
| DC 245 | 7.30 | 3.00 | 3.40 | 12.60 | 6.60 |
| Fluid AP² | | | 3.00 | | 6.00 |
| Eutanol G³ | | | 0.12 | | 0.23 |
| Triclosan⁴ | | | 0.05 | | |
| BHT | | 0.10 | | 0.50 | |
| CAP 40 | to 100 | to 100 | to 100 | to 100 | to 100 |

¹50 cs. dimethiconepolydimethylsiloxane, ex Dow Corning.
²PPG-14 butylether, ex Union Carbide.
³2-octyldodecanol, ex Cognis.
⁴2',4,4'-trichloro,2-hydroxy-diphenyl ether, ex Dastech International.

TABLE 5
Non-Aerosol Compositional Examples

| | Example: | | | |
|---|---|---|---|---|
| Components: | 10 | 11 | 12 | 13 |
| DTPA | 2.0 | 1.5 | 0.75 | 1.0 |
| Perfume | 1.0 | 1.0 | 1.0 | 1.0 |
| Fluid AP | 23.6 | | 24.0 | 25.5 |
| Stearyl alcohol | 23.6 | | | |
| Castor wax MP80¹ | 3.6 | | 2.5 | |
| PEG-8 distearate | 2.7 | | 2.5 | |
| Finsolv TN² | | 26.7 | | |
| β-sitosterol | | 4.0 | | |
| γ-oryzanol | | 6.0 | | |
| Synchrowax ERL-C³ | | | 7.5 | |
| Propylene carbonate | | | | 1.5 |
| Bentone 38V | | | | 4.5 |
| DC 245 | to 100 | to 100 | to 100 | to 100 |

1hydrogenated castor oil, ex Caschem.
2C12-C15 alkyl benzoate, ex Finetex.
3C18-C36 glycol ester, ex Croda.

Examples 10 and 11 are stick products, whilst Example 12 is a soft solid and Example 13 is a roll-on.

1. A deodorant composition comprising a suspension of a particulate transition metal chelator having a weight average particle size of between 10 μm and 100 μm in a silicone fluid carrier material, wherein the deodorant composition excludes a significant amount of astringent antiperspirant salts.

2. A deodorant composition according to claim 1 that is essentially anhydrous.

3. A deodorant composition according to claim 1, wherein the transition metal chelator has an iron binding constant of greater than 10²⁶.

4. A deodorant composition according to claim 1, wherein the transition metal chelator is in its acid form or its acid salt form.

5. A deodorant composition according to claim 3, wherein the transition metal chelator has the acid form diethylenetriaminepentaacetic acid (DTPA), triethylenetetraaminehexaacetic acid, or ethylenebis [2-(2-hydroxyphenyl)glycine].

6. A deodorant composition according to claim 5, wherein the transition metal chelator has the acid form DTPA.

7. A deodorant composition according to claim 1, comprising an amount by weight of additional components having a water absorbency of greater than 2.5 g/g that is less than the amount by weight of the transition metal chelator.

8. A deodorant composition according to claim 7, comprising an amount by weight of additional components having a water absorbency of greater than 1.5 g/g that is less than the amount by weight of the transition metal chelator.

9. A deodorant composition according to claim 7, comprising a weight ratio of the transition metal chelator to additional components having a water absorbency of greater than 2.5 g/g that is greater than 2:1.

10. A deodorant composition according to claim 8, comprising a weight ratio of the transition metal chelator to additional components having a water absorbency of greater than 1.5 g/g that is greater than 2:1.

11. A deodorant composition according to claim 1, comprising at least 0.2% by weight of the transition metal chelator and between 3% and 95% by weight of silicone fluid carrier material.

12. A deodorant composition according to claim 11, comprising from 0.65% to 3% by weight of the transition metal chelator and from 6% to 70% by weight of a silicone fluid carrier material.

13. A deodorant composition according to claim 11, that is an aerosol composition comprising a volatile propellant and from 3% to 50% by weight of the silicone fluid carrier material.

14. A deodorant composition according to claim 1, characterised in being an aerosol or roll-on composition and comprising 0.1 to 10% by weight of a suspending agent.

15. A deodorant composition according to claim 14, comprising propylene carbonate.

16. A deodorant composition according to claim 1, comprising an additional organic anti-microbial agent at a level of from 0.01% to 3% by weight.

17. A deodorant composition according to claim 1, comprising an anti-oxidant at a level of from 0.05% to 5% by weight.

18. A deodorant composition according to claim 1, comprising a fragrance material.

19. A method of achieving a deodorancy benefit comprising the application to the human body or to an article wearable in close proximity thereto of a composition according to claim 1.

20. A method for the manufacture of a deodorant composition, said method comprising the suspension of a particulate transition metal chelator of weight average particle size between 10 μm and 100 μm in a silicone fluid carrier material, without addition of a significant amount of astringent antiperspirant salts to said deodorant composition.
